(19) United States
(12) Patent Application Publication
(10) Pub. No.: US 2020/0324098 A1
(43) Pub. Date: Oct. 15, 2020

(54) Drive system and drive module for a hand-held device for locally piercing of human or animal skin, and hand-held device

(71) Applicant: MT.DERM GmbH, Berlin (DE)

(72) Inventor: Dirk Scherkowski, Berlin (DE)

(21) Appl. No.: 16/849,460

(22) Filed: Apr. 15, 2020

(30) Foreign Application Priority Data
Apr. 15, 2019 (EP) 19169338.1

(51) Int. Cl. A61M 37/00; A01K 11/00

(52) U.S. Cl. CPC A61M 37/0076; A61M 2205/103; A01K 11/005

(57) ABSTRACT

The invention relates to a drive system for a hand-held device for locally piercing human or animal skin, having a drive means which is arranged to provide a rotary drive movement; and a conversion mechanism (4b) which couples to the drive means for tapping the rotary drive movement and is arranged to provide a linear forward and backward movement starting from the rotary drive movement, the conversion mechanism (4b) having: a wobble plate arrangement arranged on a bearing means and coupling via a rotary joint to a distal joint component which is accommodated by the bearing means; a wobble plate formed on the wobble plate arrangement which, in operation, performs a wobbling motion due to the rotary driving motion; a tap, which is coupled to the wobble plate and is adjusted to provide the forward and backward linear movement for tapping by cooperation with the wobble plate; and an adjustment component, which is coupled to the wobble plate arrangement and is displaceably arranged such that for adjusting a stroke of the forward and backward linear movement provided at the tap, a tilt of the wobble plate with respect to the longitudinal direction can be changed by displacing the adjustment component. Furthermore, a drive module and a hand-held device are provided.

The invention relates to a drive system and a drive module for a hand-held device for localized piercing of human or animal skin and a hand-held device.

### BACKGROUND

Such skin piercing apparatuses are used to pierce the skin locally. Here, it can be provided to introduce a chemical into the skin by means of local piercing of the skin, for example a dye, in connection with the formation of tattoos or permanent makeup, or a cosmetic or medical substance.

Conventional skin piercing apparatuses are designed as hand-held devices, in which a drive module and a needle module coupled to it are provided. For example, the drive module has an electric motor that provides a drive movement to extend and retract a piercing needle through a front housing opening of the needle module. The drive movement provided by the drive module is coupled to the piercing needle so that it can be moved back and forth for local skin piercing. Drive module and needle module can be detachably coupled together, wherein the needle module can be designed as a disposable module. The forward and backward movement of the piercing needle takes place during operation with a machine-controlled repetition frequency.

### SUMMARY

The object of the invention is to provide a drive system as well as a drive module for a hand-held device for local piercing of human or animal skin as well as a hand-held device, which can be flexibly adapted to different application conditions.

As a solution, a drive system as well as a drive module for a hand-held device for local piercing of human or animal skin according to claims 1 and 12 have been created. Furthermore, a hand-held device for the local piercing of human or animal skin according to claim 13 has been created. Embodiments are subject to dependent claims.

According to one aspect, a drive system is created for a hand-held device for locally piercing a human or animal skin, which has a drive means which is adapted to provide a rotary drive movement, and a conversion mechanism which couples to the drive means to pick up the rotary drive movement and is adapted to provide a linear forward and backward movement starting from the rotary drive movement. The conversion mechanism has the following features: a wobble plate arrangement arranged on a bearing means and coupled via a rotary joint to a joint component, which is accommodated by the bearing means; a wobble plate formed on the wobble plate arrangement which, during operation, performs a wobbling movement due to the rotational drive movement; a tap coupled to the wobble plate and arranged to provide, by means of engaging with the wobble plate, the linear forward and backward movement for tapping; and an adjustment component coupled to the wobble plate arrangement and displaceably arranged, such that, for adjusting a stroke of the linear forward and backward movement provided at the tap, a tilt of the wobble plate to the longitudinal direction can be changed by means of displacing the adjustment component.

According to another aspect, a drive model for a hand-held device for local piercing of a human or animal skin is created with the drive system, which can be coupled to a needle module with a piercing device for local skin piercing. Furthermore, a hand-held device for local piercing of human or animal skin is created with the drive module and a needle module coupled to it.

With the help of the proposed drive system, the stroke of the linear forward and backward movement can be flexibly provided for various applications for local skin piercing. In the handheld device for local skin piercing, which can also be called a skin piercing device, the linear forward and backward movement provided by the drive system is coupled to the piercing means, which is then moved back and forth. Using the proposed drive system, a flexibly adjustable stroke can be provided for linear forward and backward movement. For this purpose, the adjustment component, which couples to the wobble plate arrangement, is displaceable, for example pivotable, to adjust the tilt or inclination of the wobble plate to the longitudinal direction, i.e. an angle of attack. This causes a change in the stroke of the linear forward and backward movement provided by the conversion mechanism, which can be tapped on the hand-held device for local skin piercing for coupling to the piercing means.

To change the tilt of the wobble plate to the longitudinal direction by means of displacing the adjustment component, a storage of the adjustment component can be displaced in the longitudinal direction.

The drive means can be formed using an electric motor. The repetition frequency at which the linear forward and backward movement is provided can be between about 0.5 and about 200 Hz, alternatively between about 10 and about 200 Hz or between about 40 and about 200 Hz.

In the case of the hand-held device, the needle module with associated piercing means, which is known as such in various embodiments, can be designed as a disposable module. In the needle module, one or more components can be made of a plastic material, for example by injection molding. Especially in the configuration as a disposable module, the needle module is detachably arranged on the drive module. With the help of the drive module, the linear forward and backward movement is provided, which is tapped as drive movement on the needle module's piercing means in order to be able to perform repeated local skin piercing.

The rotary joint can be designed as a ball joint, for example.

The adjustment component can be pivotally stored on the wobble plate arrangement. For example, the bearing of the adjustment component on the wobble plate arrangement can be formed by means of a bearing journal and an associated bearing opening in which the bearing journal is located. The adjustment component can then be pivotably stored in opposite end regions of the adjustment component.

The adjustment component can be formed with a flat component, the flat sides of which extend in the longitudinal direction. The flat component can be formed with a disk component. In one embodiment, the flat component is pivotally or rotatably mounted on the component storage and on the wobble plate arrangement.

The adjustment component can be arranged displaceably on a component bearing, for example pivotably, in such a way that for adjusting the stroke of the linear forward and backward movement provided at the tap, the tilt of the wobble plate to the longitudinal direction can be changed by displacing the adjustment component, wherein for this purpose the component bearing can be displaced in the longitudinal direction.

The adjustment component can be arranged pivotally on the component bearing in such a way that the tilt of the wobble plate to the longitudinal direction can be changed by pivoting the adjustment component in order to adjust the stroke of the linear forward and backward movement provided at the tap.

The adjustment component can be arranged such that it can be displaced by means of a solid-state joint. For adjusting the stroke of the linear forward and backward movement provided at the tap, the tilt of the wobble plate to the longitudinal direction can be changed by displacing the adjustment component, wherein for this purpose a bearing of the solid-state joint can be displaced in the longitudinal direction.

The component bearing can be displaceable while changing a distance between a rotary joint center of the rotary joint and the component bearing. If the rotary joint is designed as a ball joint, the ball head can be formed in one piece with the bearing component accommodated in the bearing means, for example in a distal bearing component. In this example, the wobble plate arrangement is movably accommodated at the ball head according to the degrees of freedom of a ball joint. With this or other embodiments, the wobble plate arrangement can have an inner component which sits on the ball head. A portion of the inner component accommodating the ball head can couple to the wobble plate by means of a rotary decoupling, which can be formed with a ball bearing.

When adjusting the tilt of the wobble plate to the longitudinal direction, which in turn may coincide with an axial direction of a drive shaft of the drive means, a plane transverse to the longitudinal direction is more or less tilted, which extends through the center of the rotary joint, in particular the ball head center of the ball joint.

The component bearing can have a bearing journal, which is rotatably arranged in an associated bearing bush. The bearing bush can be provided on the adjustment component.

The component bearing can be displaced in the longitudinal direction following a displacement of the drive means. A displacement of the drive system, for example an electric motor comprised thereof with drive shaft, in the longitudinal direction causes or forces a displacement, for example (shifting) of the component bearing, so that the tilt of the wobble plate is altered. In this case, the component bearing and the drive means can be connected to each other at a fixed distance in the longitudinal direction, for example in such a way that a component seated on the drive shaft of the drive means provides or firmly couples the component bearing to which the adjusting component is pivotably coupled.

The component bearing can be arranged on a component that can be displaced in the longitudinal direction relative to the drive means, for example in a housing that accommodates the drive means. In this embodiment, the drive means itself can be stationary arranged. Relative to the drive means the displaceable component is displaced in order to displace the component bearing in longitudinal direction, in particular to shift it in order to adjust the tilt of the wobble plate. The displaceable component, on which the component bearing is formed, can perform a relative displacement in relation to the bearing means.

The drive means and the conversion mechanism can be arranged in a housing, wherein the bearing means is arranged in a fixed position in the housing.

The bearing means can comprise a bearing means proximal to the drive means and a bearing means distal to the drive means and spaced apart from the proximal bearing means in a longitudinal direction. If the bearing means are provided stationary in the housing, the distance in the longitudinal direction between the proximal and the distal bearing means is fixed. Such configuration can also be provided in other embodiments.

The wobble plate arrangement can be placed between the proximal and the distal bearing means.

The longitudinal direction may coincide with the axial direction of the drive shaft.

The precedingly described configurations can be provided for the drive module as well as for the hand-held device.

### DESCRIPTION OF EMBODIMENTS

In the following, further embodiments are explained in more detail with reference to the drawings. Here:

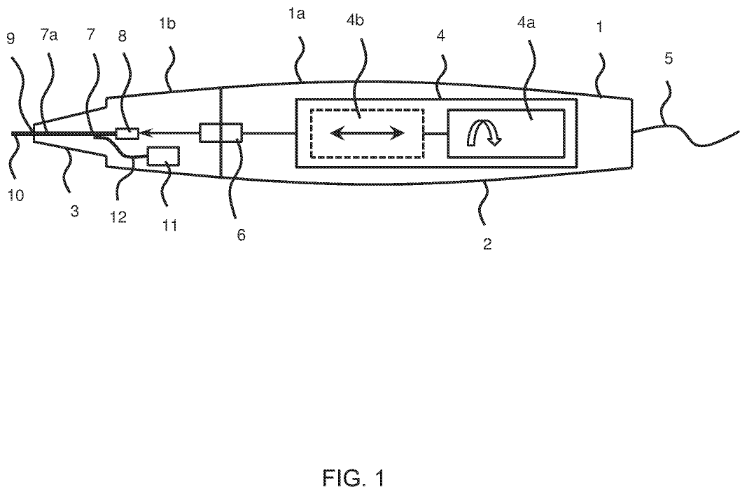

FIG. 1 shows a schematic representation of a hand-held device for local piercing of human or animal skin with a drive module and a needle module coupled to it;

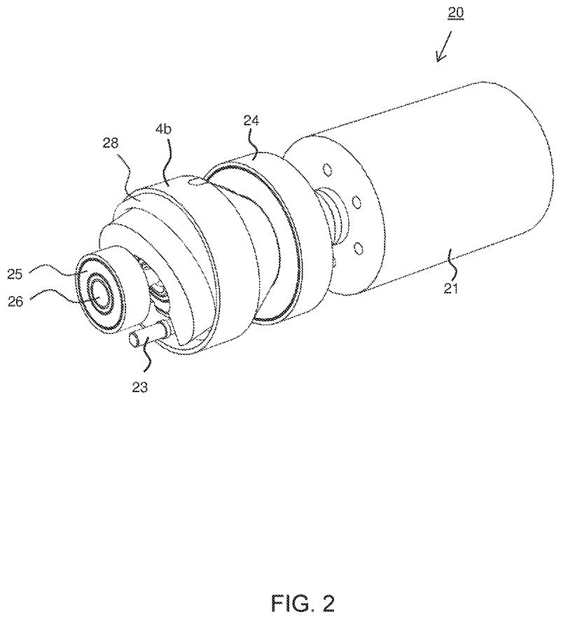

FIG. 2 shows a perspective representation of a drive system for the hand-held device;

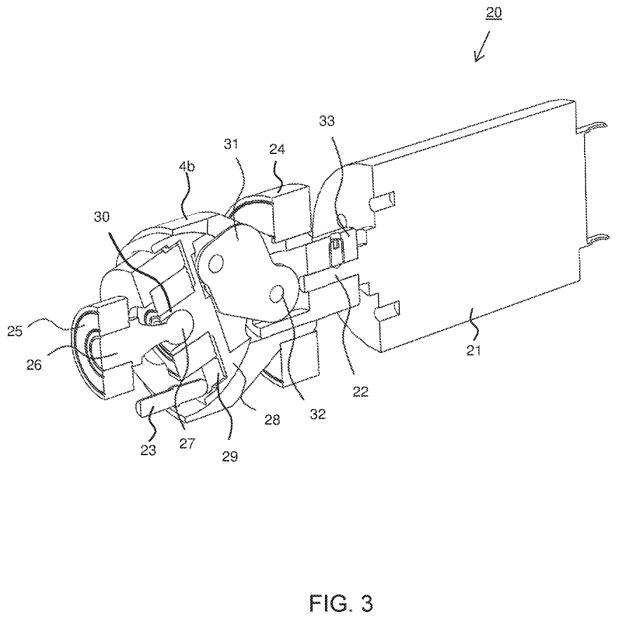

FIG. 3 shows a perspective representation of the drive system from FIG. 2 in the section;

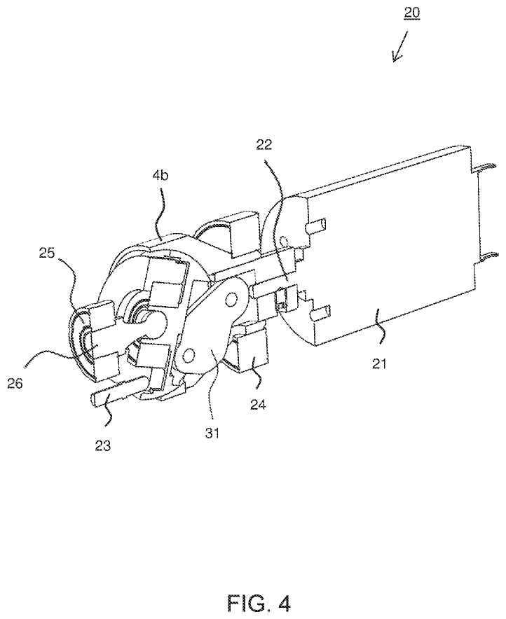

FIG. 4 shows a perspective representation of the drive system from FIG. 3 with a different operating position in the section;

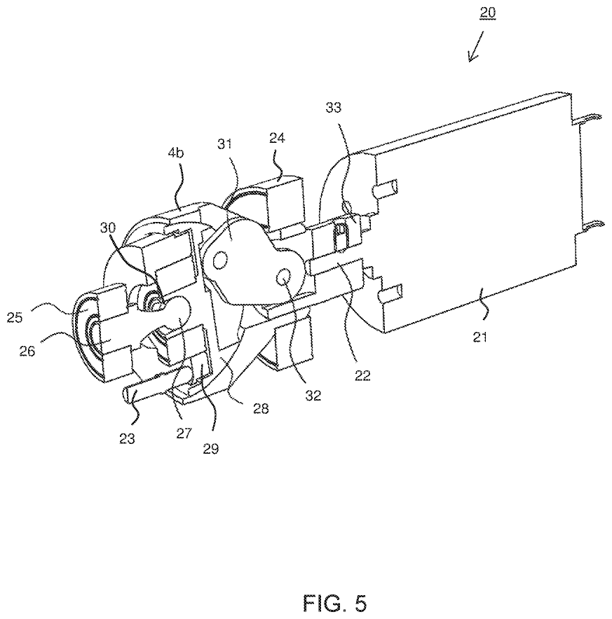

FIG. 5 shows a perspective representation of the drive system from FIG. 3 with a further operating position in the section;

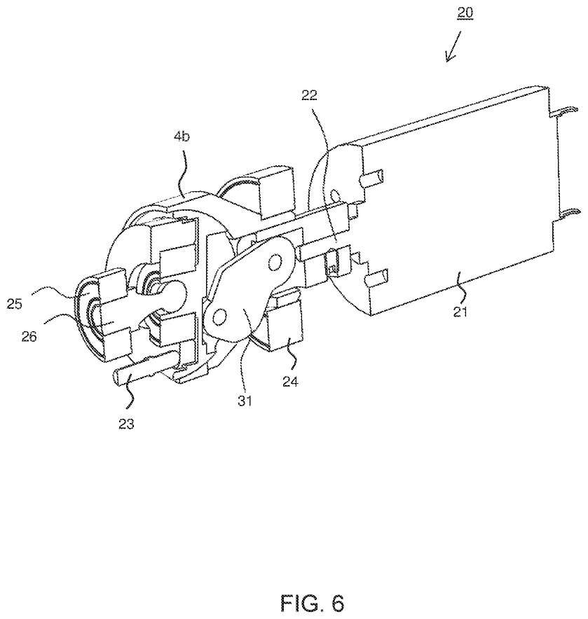

FIG. 6 shows a perspective representation of the drive system from FIG. 3 with another changed operating position in the section;

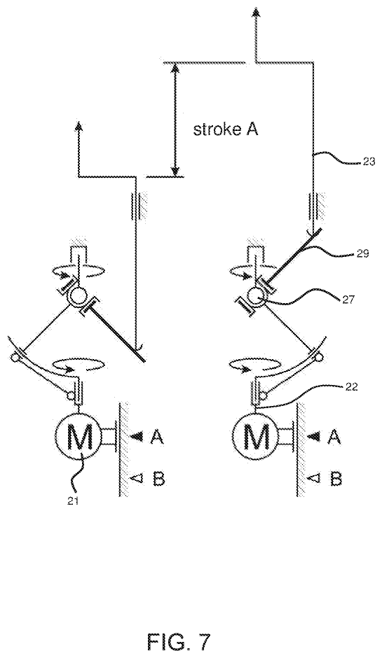

FIG. 7 shows a schematic functional representation for members of the drive system from FIG. 3 and FIG. 4 with a stroke adjustment A;

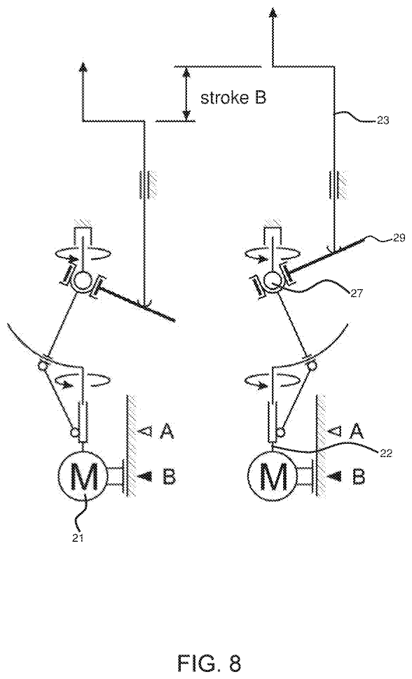

FIG. 8 shows a schematic functional representation for members of the drive system from FIG. 5 and FIG. 6 with a stroke adjustment B;

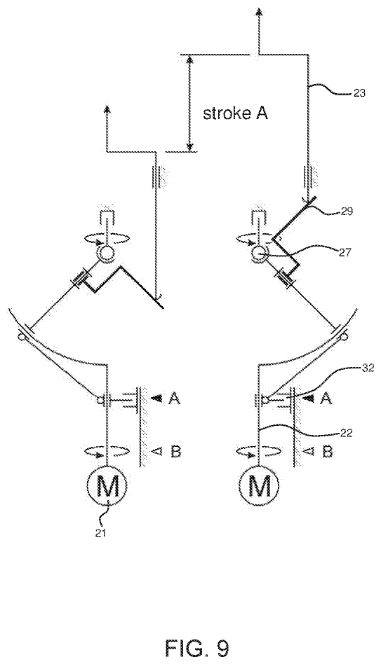

FIG. 9 shows a schematic functional representation for members of another configuration of a drive system with a stroke adjustment A, and

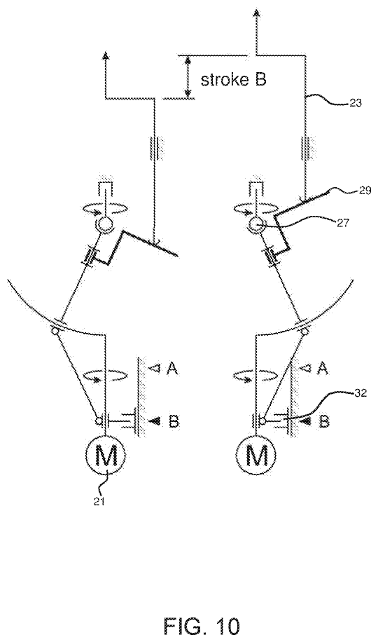

FIG. 10 shows a schematic functional representation for members of the other configuration according to FIG. 9 with a stroke adjustment B which is different from stroke adjustment A.

FIG. 1 shows a schematic representation of a hand-held device for the repeated local piercing of human or animal skin (skin piercing device) with a housing 1. In the example shown, housing 1 is modularly built with a drive module housing 1a of a drive module 2 and a needle module housing 1b of a needle module 3, which is detachably coupled to drive module 2.

A drive system 4 is provided in the drive module housing 1a, which has a drive means 4a with a motor and a conversion mechanism 4b in the represented embodiment. Using the motor in the example, a rotary drive movement (drive force) is provided, which is converted, by means of the conversion mechanism 4b, into a drive force acting in a linear direction in the longitudinal direction (back and forth movement) of housing 1, whether for carrying out a single stroke or for a repetitive movement.

For power supply, the hand-held device can be connected to a supply voltage via a feed line 5. The feed line 5 can additionally comprise cables through which control signals are transmitted between a control device (not shown) and the hand-held device.

The driving force generated mechanically by the drive system 4 is transmitted via a coupling means 6 to a piercing means 7 having a piercing tool 7a, which is arranged in a tool holder 8. When using one or more needles as a piercing tool 7a, the tool holder 8 can be a needle shank arranged in the needle module housing 1b in an associated guide so that the repetitive or cyclical drive force can be converted in a linear direction for forward and backward movement of tool holder 8 with piercing tool 7a. The piercing tool 7a is passed through a housing opening 9 at the front of the needle module housing 1b, so that at least in one extended position a piercing tool tip 10 is arranged outside the needle module housing 1b to pierce the skin to be treated. The housing opening 9 in the shown embodiment is formed at the front end of the needle module housing 1b on the needle module 3, which is detachably mounted on the drive module housing 1a.

In the shown embodiment of the hand-held device, a reservoir 11 is also provided in housing 1, which serves to accommodate a chemical to be introduced into the skin, for example a color in the case of producing a tattoo or permanent make-up. The chemical is fed to the piercing tool 7a via an outlet 12, in order to provide the chemical at the piercing tool tip 10 for introducing it into the skin. Alternatively, the reservoir 11 can also be arranged in the drive module housing 1a or outside the housing 1. Various configurations for such a reservoir and the provision of the chemical to be introduced in the area of the piercing tool 7 are known as such, for example, and also with the use of a pumping means on the reservoir 11.

FIG. 2 shows a schematic perspective representation of a drive system 20, which can be used for drive module 2, for example. FIGS. 3 to 6 show respective sectional representations of the drive system 20 with different operating positions.

With the aid of an electric motor 21, a rotary drive movement is provided via a drive shaft 22, which is tapped off by the conversion mechanism 4b and converted into a linear forward and backward movement, which can be tapped off via a tap 23, in order to couple this linear forward and backward movement as a drive movement to a piercing means of the needle module 3. The tap 23 can be part of the coupling means 6.

In the conversion mechanism 4b, a proximal and a distal bearing means 24, 25 are provided in relation to the electric motor 21, which in one embodiment can be mounted stationary or fixedly in a housing of the drive module 2. A bearing component 26 with a ball head 27, which is integrally formed in the example shown, is mounted on the distal bearing means 25. On the ball head 27 rests a wobble plate arrangement 28 with a wobble plate 29. The ball head 27 forms a ball joint together with an inner component 30 of the wobble plate arrangement 28.

The wobble plate arrangement 28 is coupled to an adjustment component 31 which is pivotally mounted on the wobble plate arrangement 28 as well as on a component bearing 32. In the represented embodiment, the adjustment component 31 is formed with a disk or flat component. The component bearing 32 is firmly connected to the drive shaft 22 via a coupling component 33. Due to the rotating drive movement which is provided by the drive shaft 22, the coupling component 33 as well as the adjustment component 31 and parts of the wobble plate arrangement 28 rotate relative to the proximal and distal bearing means 24, 25, causing the wobble plate 29 to perform a wobbling movement that allows the tapping off of the linear back and forth movement at the tap 23.

By displacing the component bearing 32 in longitudinal direction the distance of the component bearing 32 to the center of the ball head 27 can be changed, which causes a changing of the tilt or inclination of the wobble plate 29 to the longitudinal direction, so that a stroke of the linear forward and backward movement can be adjusted.

Different configurations can be provided for displacing the component bearing 32 in the longitudinal direction. In an example, the electric motor 21 can be displaced in longitudinal direction. Via the drive shaft 22 and the coupling component 33 which is firmly connected to it, the component bearing 32 is then also displaced in the longitudinal direction. Alternatively, the electric motor 21 can be fixedly accommodated in the housing of drive module 2. The coupling component 33, which is non-rotatably connected to the drive shaft 22, can then be moved in the longitudinal direction to shift the component bearing 32. For this purpose, a drive (not represented) can be provided to displace the coupling component 33.

FIGS. 7 and 8 show schematic functional representations for members of the drive system from FIGS. 2 to 6 with different stroke adjustments A, B. The same reference signs as in FIGS. 2 to 6 are used for the same features. In FIGS. 7 and 8 two different rotary positions for the wobble plate 29 are shown, whose wobbling movement leads to stroke A (FIG. 7) or stroke B (FIG. 8) for the linear forward and backward movement. The different strokes are caused by the different inclinations of the wobble plate 29. In the example shown, the electric motor 21 is displaced (motor shift).

FIGS. 9 and 10 show schematic functional representations for members of another configuration with different stroke adjustments A, B. The same reference signs as in FIGS. 2 to 6 are used for the same features. In FIGS. 9 and 10 two different rotary positions for the wobble plate 29 are shown, whose wobbling movement leads to stroke A (FIG. 9) or stroke B (FIG. 10) for the linear forward and backward movement. The different strokes are caused by the different inclinations of the wobble plate 29. In the alternative example shown, the electric motor 21 remains stationary if the component bearing 32 (relative to the ball head 27) is moved for stroke adjustment. For implementation, for example, a housing/adjustment sleeve with a circumferential groove on the inside can be provided which can be shifted in the axial direction, in which a shift cam on a rotating component revolves in the groove and is displaced relative to the rotating component by means of sleeve displacement.

The features disclosed in the above-mentioned description, claims and drawing can be relevant to the realization of the different configurations either individually or in any combination.

1. A drive system for a hand-held device for locally piercing a human or animal skin, having:
a drive means adapted to provide a rotary drive movement; and
a conversion mechanism, which is coupled to the drive means for tapping the rotary drive movement and is adapted to provide a linear forward and backward movement starting from the rotary drive movement, the conversion mechanism having:
a wobble plate arrangement arranged on a bearing means and coupled via a rotary joint to a distal joint component received by the bearing means;
a wobble plate formed on the wobble plate arrangement, which during operation performs a wobbling motion due to the rotating drive motion;
a tap, which is coupled to the wobble plate and adapted to provide linear forward and backward movement for tapping by engaging with the wobble plate; and
an adjustment component which is coupled to the wobble plate arrangement and displaceably arranged such that for adjusting a stroke of the linear forward and backward movement provided at the tap, a tilt of the wobble plate with respect to the longitudinal direction can be changed by displacing the adjustment component.

2. The drive system according to claim 1, wherein the adjustment component is pivotally mounted on the wobble plate arrangement.

3. The drive system according to claim 1, the adjustment component is formed using a flat component whose flat sides extend in the longitudinal direction.

4. The drive system according to claim 1, wherein the adjustment component is displaceably arranged on a component bearing in such a way that for adjusting the stroke of the linear forward and backward movement provided at the tap the tilt of the wobble plate, relative to the longitudinal direction, can be changed by means of displacing the adjustment component, wherein for this purpose, the component bearing being displaceable in the longitudinal direction.

5. The drive system according to claim 4, wherein the component bearing can be displaced in such a way as to change a distance between a rotary joint center of the rotary joint and the component bearing.

6. The drive system according to claim 4, wherein the component bearing has a bearing journal which is rotatably arranged in an associated bearing bush.

7. The drive system according to claim 4, wherein the component bearing can be displaced following a displacement of the drive means in the longitudinal direction.

8. The drive system according to claim 4, wherein the component bearing is arranged on a component which is displaceable in the longitudinal direction relative to the drive means.

9. The drive system according to claim 1, wherein the drive means and the conversion mechanism (4b) are arranged in a housing, the bearing means being arranged fixedly in the housing.

10. The drive system according to claim 1, wherein the bearing means has a bearing means which is proximal with respect to the drive means and a bearing means which is distal with respect to the drive means and is spaced apart from the proximal bearing means in a longitudinal direction.

11. The drive system according to claim 10, wherein the wobble plate arrangement is arranged between the proximal and distal bearing means.

12. A drive module for a hand-held device for locally piercing a human or animal skin, which has a drive system according to claim 1 and can be coupled to a needle module having a piercing means for local skin piercing.

13. A hand-held device for locally piercing a human or animal skin, having a drive module according to claim 12 and a needle module coupled thereto with a piercing means for locally piercing the skin.
